(19) United States
(12) Patent Application Publication
(10) Pub. No.: US 2019/0104922 A1
(43) Pub. Date: Apr. 11, 2019

(54) WIRELESS ENDOSCOPE APPARATUS

(71) Applicant: OLYMPUS CORPORATION, Tokyo (JP)

(72) Inventor: Makoto KASUMI, Hachioji-shi (JP)

(73) Assignee: OLYMPUS CORPORATION, Tokyo (JP)

(21) Appl. No.: 16/211,124

(22) Filed: Dec. 5, 2018

Related U.S. Application Data
(63) Continuation of application No. PCT/JP2017/008681, filed on Mar. 6, 2017.

(30) Foreign Application Priority Data
Sep. 29, 2016 (JP) 2016-190792

(51) Int. Cl. A61B 1/00; A61B 50/22; A61B 1/05; A61B 1/06

(52) U.S. Cl. CPC A61B 1/00016; A61B 1/00006; A61B 1/00032; A61B 1/00108; A61B 1/00036; H04N 2005/2255; A61B 1/00045; A61B 1/05; A61B 1/0676; A61B 1/0684; A61B 50/22

(57) ABSTRACT

A wireless endoscope system includes two wireless endoscopes and a processor. Each wireless endoscope includes a memory configured to store setting information specifying operation conditions. The processor is capable of performing radio communication with each wireless endoscope. The processor includes a memory configured to store the setting information acquired by radio communication, and a controlling portion. The controlling portion stores the setting information in the memory and transmits the setting information to one of the wireless endoscopes by near field radio communication to store the setting information in a memory of the one of the wireless endoscope.

### CROSS REFERENCE TO RELATED APPLICATION

This application is a continuation application of PCT/JP2017/008681 filed on Mar. 6, 2017 and claims benefit of Japanese Application No. 2016-190792 filed in Japan on Sep. 29, 2016, the entire contents of which are incorporated herein by this reference.

### BACKGROUND OF INVENTION

### 1. Field of the Invention

The present invention relates to a wireless endoscope apparatus, and in particular to a wireless endoscope apparatus including wireless endoscopes that can be driven by batteries.

### 2. Description of the Related Art

Conventionally, downsizing and power consumption reduction of various apparatuses, such as a mobile phone, a smartphone and a tablet PC, have been promoted due to progress of semiconductor technology, and the apparatuses are portably configured. A mobile apparatus is often configured to be equipped with a battery so that the mobile apparatus can be continuously used by charging the battery.

In a medical field, downsizing of apparatuses has also been promoted, and a wireless endoscope system is also proposed as disclosed in Japanese Patent Application Laid-Open Publication No. 2010-207459. Furthermore, even among endoscopes with relatively high power consumption, wireless endoscopes equipped with a chargeable battery have been developed.

Wireless endoscopes are used in various fields, for example, the medical field and an industrial field. A wireless endoscope in the medical field is used for observation of an organ in a body cavity, medical care and treatment using a treatment instrument, a surgical operation under endoscope observation and the like.

Since a wireless endoscope does not have a cable to be connected to a video processor, it is easy for a surgeon to handle the wireless endoscope.

An image signal of a picked-up image obtained by a wireless endoscope is transmitted by radio to a processor configured to perform image processing. The processor performs display of a medical image on a monitor and recording of the medical image to a recording medium. The wireless endoscope is excellent in portability and workability by being wirelessly configured, including a radio communication portion for transmitting an endoscopic image obtained by an image pickup device to the processor, a light source apparatus for illuminating an object and the like.

Various settings for the wireless endoscope are changed before or during a surgical operation. For example, there may be a case where settings for a radio channel, an observation mode and the like are changed. Information about the changed settings is held by the wireless endoscope together with setting information and, if necessary, is also transmitted to the processor by radio and held by the processor.

When, in a case where the wireless endoscope is of a type that a battery is embedded inside in a manner that the battery cannot be exchanged, the battery runs out during a surgical operation, the surgeon who is a user pulls the wireless endoscope being used out of a body cavity, exchanges the wireless endoscope for another charged wireless endoscope, and then performs examination or the like again. pe, the exchanged other wireless endoscope to be usable in a short time period.

### SUMMARY OF THE INVENTION

A wireless endoscope apparatus of an aspect of the present invention includes: a first wireless endoscope that can be driven by a first battery; a second wireless endoscope that can be driven by a second battery; a receiver capable of radio communication with the first wireless endoscope and the second wireless endoscope; a first memory provided in the first wireless endoscope and configured to store setting information specifying operation condition of the first wireless endoscope; a second memory provided in the second wireless endoscope and being capable of storing the setting information; a third memory provided in the receiver and being capable of storing the setting information; and a control device provided in the receiver and configured to receive the setting information stored in the first memory from the first wireless endoscope that is in an operating state in which it is possible to pick up an image, via the radio communication and store the setting information in the third memory, and, while the first wireless endoscope is in the operating state, transmit the setting information to the second wireless endoscope that is in a standby state different from the operating state so that the setting information is stored in the second memory.

### BRIEF DESCRIPTION OF THE DRAWINGS

FIG. 1 is an explanatory diagram showing an overall configuration of an endoscope system disposed in an operating room according to an embodiment of the present invention;

FIG. 2 is a block diagram showing a configuration of a wireless endoscope according to the embodiment of the present invention;

FIG. 3 is a block diagram showing a configuration of a processor according to the embodiment of the present invention;

FIG. 4 is a partial perspective view for illustrating positions of the wireless endoscope hung on a hanger, an antenna for near field radio communication of the hanger, and a power supply element for power supply according to the embodiment of the present invention;

FIG. 5 is a state transition diagram for illustrating operation modes of the wireless endoscope according to the embodiment of the present invention;

FIG. 6 is a flowchart showing an example of a flow of an operation of the wireless endoscope when a power source of the wireless endoscope is turned on, and the wireless endoscope enters an operating state, according to the embodiment of the present invention;

FIG. 7 is a flowchart showing an example of a flow of a setting information transmission process of the processor when setting information about the wireless endoscope that is in the operating state is changed, according to the embodiment of the present invention; and

FIG. 8 is a flowchart showing an example of a flow of an operation of the wireless endoscope that is in a standby state, according to the embodiment of the present invention.

### DETAILED DESCRIPTION OF THE PREFERRED EMBODIMENT(S)

An embodiment of the present invention will be described below with reference to drawings.

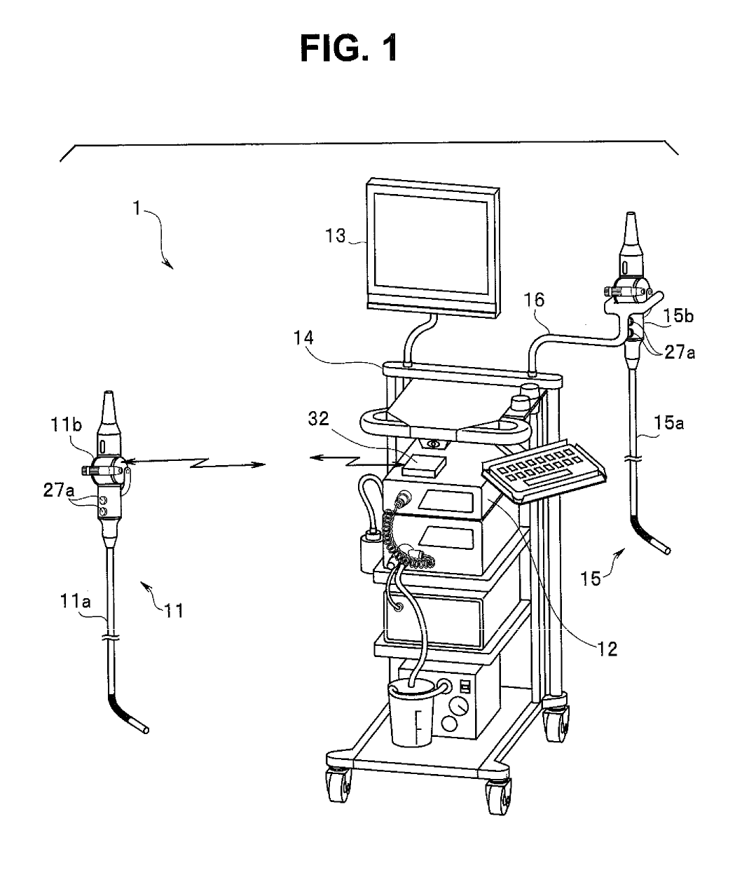

FIG. 1 is an explanatory diagram showing an overall configuration of an endoscope system disposed in an operating room according to the present embodiment.

As shown in FIG. 1, an endoscope system 1 is configured, including a wireless endoscope 11, a processor 12 and a monitor 13. In an operating room, the processor 12, the monitor 13 and various medical apparatuses are mounted on a cart 14. The processor 12 includes a radio communication portion 32. As the various medical apparatuses, for example, apparatuses such as an electric knife apparatus, a pneumoperitoneum apparatus and a video recorder, a gas cylinder filled with carbon dioxide, and the like are placed on the cart 14.

The cart 14 is provided with a hanger 16, which is a holding member for hanging a spare wireless endoscope 15. The hanger 16 constitutes a holding portion configured to hold the spare wireless endoscope 15.

The spare wireless endoscope 15 serves as a backup in case a battery of the wireless endoscope 11 runs out. In the case of FIG. 1, the wireless endoscope 11 is an endoscope that a surgeon uses first, and the wireless endoscope 15 hung on the hanger 16 is another wireless endoscope to be exchanged for the wireless endoscope 11 and used when the battery of the wireless endoscope 11 runs out.

As described above, the endoscope system 1 constitutes a wireless endoscope apparatus that includes the wireless endoscopes 11 and 15 and the processor 12.

Note that though one spare wireless endoscope is provided here, a plurality of spare wireless endoscopes may be provided. In that case, the hanger 16 is configured so that the plurality of spare wireless endoscopes can be hung.

The wireless endoscope 11 includes an insertion portion 11a on a distal end side and an operation portion 11b on a proximal end side. The insertion portion 11a includes a distal end rigid portion, a bending portion and a flexible tube portion from the distal end side. The operation portion 11b is provided with a bending operation member and various operation buttons 27a. The various operation buttons 27a are operating devices such as a freeze button operated by a user.

The wireless endoscope 15 includes an insertion portion 15a on a distal end side and an operation portion 15b on a proximal end side. The insertion portion 15a includes a distal end rigid portion, a bending portion and a flexible tube portion from the distal end side. The operation portion 15b is also provided with a bending operation member and various operation buttons 27a. The various operation buttons 27a are operating devices such as a freeze button operated by the user.

Each of the wireless endoscopes 11 and 15 is an endoscope that can be driven by batteries. The processor 12 is a video processor capable of performing radio communication with the wireless endoscopes 11 and 15, and is configured to receive an image signal from the wireless endoscopes 11 and 15 and generate an endoscopic image to be displayed on the monitor 12.

By turning on a power source switch (not shown) of the wireless endoscope 11 and turning on a power source switch (not shown) of the processor 12, the surgeon can perform endoscopic examination and the like using the wireless endoscope 11. Note that the wireless endoscope 15 hung on the hanger 16 is in the standby state, with a power source switch of the wireless endoscope 15 being off.

In the operating state, the wireless endoscope 11 wirelessly transmits an image signal of an endoscopic image acquired by an image pickup portion by radio to the processor 12 that is a receiver by radio. The processor 12 performs image processing for the image signal to generate an endoscopic image, and displays the endoscopic image on the monitor 13.

The spare wireless endoscope 15 is charged when the spare wireless endoscope 15 is hung and held on the hanger 16.

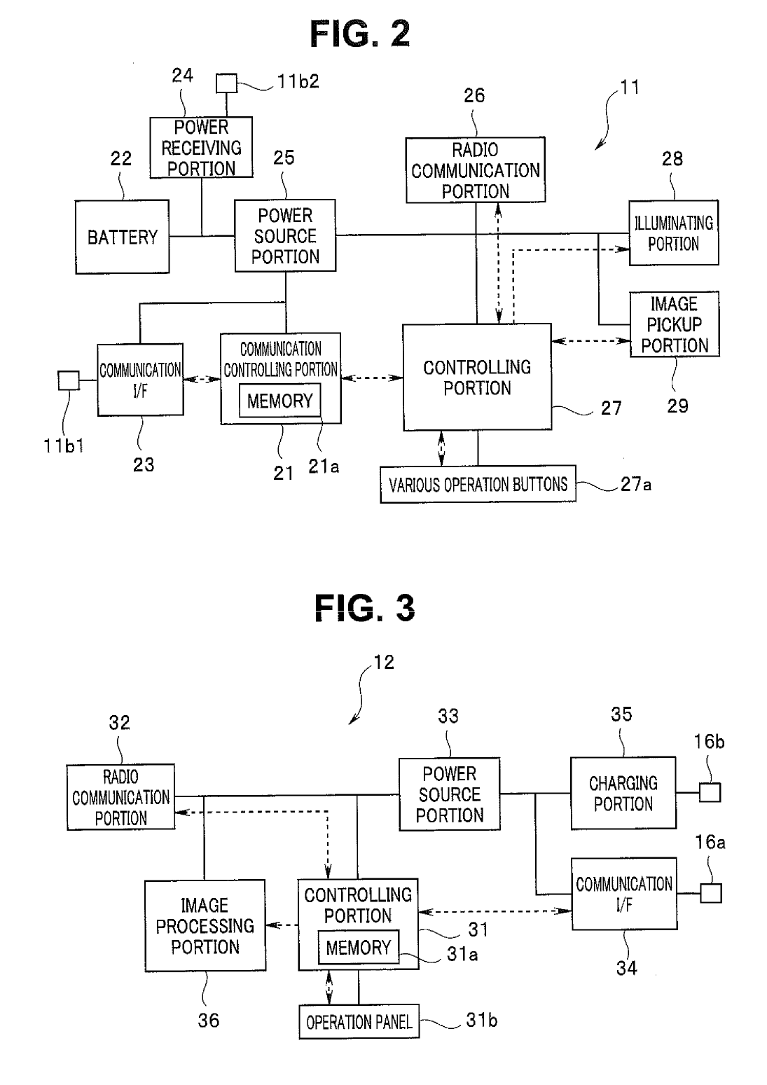

FIG. 2 is a block diagram showing a configuration of the wireless endoscope.

Though FIG. 2 shows a configuration of the wireless endoscope 11, the wireless endoscope 15 has a similar configuration.

The wireless endoscope 11 includes a communication controlling portion 21, a battery 22, a communication interface (hereinafter abbreviated as I/F) 23, a power receiving portion 24, a power source portion 25, a radio communication portion 26, a controlling portion 27, an illuminating portion 28 and an image pickup portion 29. The various operation buttons 27a are connected to the controlling portion 27.

In FIG. 2, solid lines indicate power supply lines, and dotted lines indicate control signal/data signal supply lines.

The communication controlling portion 21 includes a central processing unit (hereinafter referred to as a CPU), a ROM and a RAM. The communication controlling portion 21 monitors a state of the battery 22 and performs control to receive setting information by radio via the communication I/F 23 as described later.

The communication controlling portion 21 further includes a memory 21a configured to store setting information specifying operation conditions of the wireless endoscope 11. The operation conditions are a radio channel used for radio communication with the processor 12, settings for allocation of functions to the plurality of operation buttons provided on the operation portion 11b, and the like. That is, the memory 21a constitutes a storage portion configured to store the setting information specifying the operation conditions of the wireless endoscope 11.

The battery 22 is a chargeable secondary battery.

The communication I/F 23 is a circuit for radio communication, for example, for performing near field radio communication. An antenna 11b1 is connected to the communication I/F 23.

The power receiving portion 24 is a circuit configured to receive power supply by radio and is a circuit configured to charge the battery 22 when receiving power supply. A power receiving element 11b2 such as a coil is connected to the power receiving portion 24.

The power source portion 25 is a circuit configured to convert a power of the battery 22 to various voltages and supply the voltages to various circuits.

The radio communication portion 26 includes an antenna and is a circuit for performing radio communication using a predetermined frequency band.

The controlling portion 27 includes a CPU, a ROM and a RAM and also includes a circuit configured to control each circuit in the wireless endoscope 11 and perform image processing for an image signal from the image pickup portion 29.

When the surgeon operates the various operation buttons 27a, an operation signal or a setting signal is outputted to the controlling portion 27.

The illuminating portion 28 includes a light emitting element such as an LED, which is provided on a distal end portion of the insertion portion 11a, and a driving circuit configured to drive the LED or the like. Light generated by the illuminating portion 28 is radiated to an object as an illuminating light via an illuminating lens at a distal end of the insertion portion 11a.

The image pickup portion 29 is provided on the distal end portion of the insertion portion 11a and includes an image pickup device such as a CMOS sensor. The image pickup portion 29 is a circuit that includes an image sensor configured to receive light that has passed through an objective optical system not shown and perform photoelectric conversion.

The controlling portion 27 performs control to drive the illuminating portion 28 to cause illuminating light to be emitted from the distal end portion of the insertion portion 11a, process an image signal obtained by performing image pickup by the image pickup portion 29 and transmit the image signal to the processor 12 from the radio communication portion 26 by radio.

The controlling portion 27 also performs a process for transmitting setting information which has been changed during use to the processor 12 as described later.

FIG. 3 is a block diagram showing a configuration of the processor 12.

The processor 12 includes a controlling portion 31, the radio communication portion 32, a power source portion 33, a communication I/F 34, a charging portion 35 and an image processing portion 36. An operation panel 31b, which is an operation device operated by the user, is connected to the controlling portion 31 as a control device.

In FIG. 3, solid lines indicate power supply lines, and dotted lines indicate control signal/data signal supply lines.

The controlling portion 31 is a circuit configured to control each circuit in the processor 12 and to receive an operation signal corresponding to an operation performed on the operation panel 31b and perform a process corresponding to the operation signal.

The controlling portion 31 includes a CPU, a ROM storing various programs for realizing various functions of the processor 12 and data, and a RAM as a working memory. Furthermore, the controlling portion 31 also includes a memory 31a configured to store setting information about the wireless endoscope 11 acquired by radio communication. That is, the memory 31a constitutes a storage portion configured to store the setting information about the wireless endoscope 11 acquired by radio communication.

Furthermore, the controlling portion 31 performs control to transmit the setting information via the communication I/F 34 by radio.

The radio communication portion 32 includes an antenna and is a circuit for performing radio communication with the radio communication portion 26 of the wireless endoscope 11. That is, the wireless endoscope 11 includes the radio communication portion 26 for transmitting an image signal of an endoscopic image acquired by the wireless endoscope 11 to the processor 12 by radio communication. The processor 12 includes the radio communication portion 32 for receiving the image signal from the wireless endoscope 11 by radio communication.

The power source portion 33 is a circuit configured to generate various types of power for the respective circuits in the processor 12 and supply the various types of power to the various circuits.

The communication I/F 34 is a circuit for performing near field radio communication with the communication I/F 23 of the wireless endoscope 15 hung on the hanger 16. An antenna 16a to be described later is connected to the communication I/F 34.

That is, the processor 12 includes the communication I/F 34 as a transmitting portion for transmitting setting information to the wireless endoscope 15 by radio, and the wireless endoscope 15 includes the communication I/F 23 as a receiving portion for receiving the setting information from the processor 12 by radio.

The charging portion 35 is a circuit for charging the battery 22 of the wireless endoscope 15 hung on the hanger 16. A power supply element 16b to be described later is connected to the charging portion 35. That is, the charging portion 35 charges the battery 22 when the wireless endoscope 15 that is in the standby state is held by the hanger 16.

The image processing portion 36 is a circuit configured to perform various types of image processing for an image signal received from the wireless endoscope 11 to generate an endoscopic image.

The image processing portion 36 outputs the generated endoscopic image to the monitor 13 from an output terminal not shown.

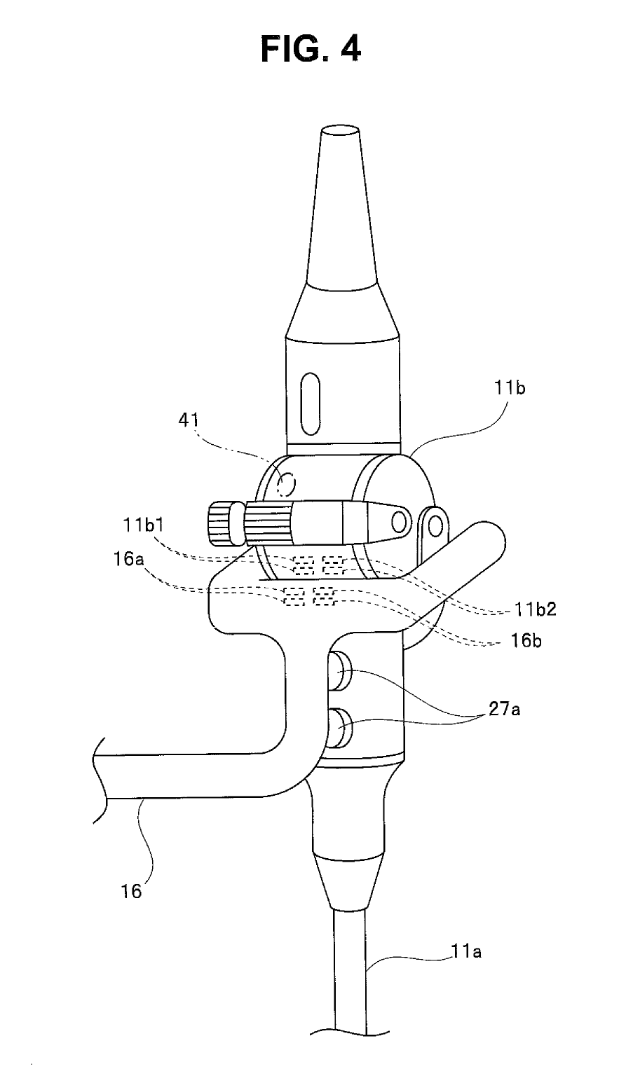

FIG. 4 is a partial perspective view for illustrating positions of the wireless endoscope 15 hung on the hanger 16, the antenna 16a for near field radio communication and the power supply element 16b for power supply of the hanger 16.

The antenna 11b1 for radio communication and the power receiving element 11b2 for power receiving such as a coil are included inside the operation portion 11b.

The antenna 16a for radio communication and the power supply element 16b for power supply such as a coil are included in the hanger 16. When the wireless endoscope 15 is hung on the hanger 16 and set, power is supplied by radio from the power supply element 16b to the power receiving element 11b2, and the antenna 16a and the power supply element 16b are arranged on the hanger 16 at positions where a near field radio signal from the antenna 16a can be appropriately received by the antenna 11b1.

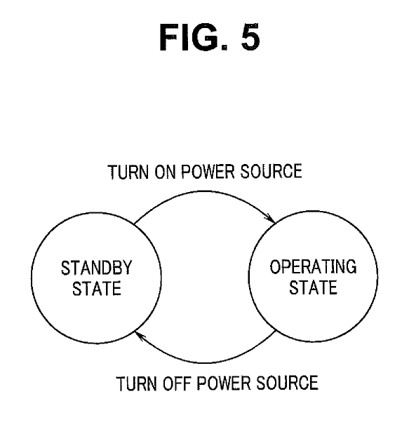

FIG. 5 is a state transition diagram for illustrating operation modes of the wireless endoscope.

The wireless endoscopes 11 and 15 can be in two operation states, that is, the standby state and the operating state. When the power source switches (not shown) provided on the wireless endoscopes 11 and 15, respectively, are off without being turned on, the wireless endoscopes 11 and 15 are in the standby state. When the power source switches (not shown) are turned on, the wireless endoscopes 11 and 15 enter the operating state.

The standby state is, in FIG. 1, the state of the wireless endoscope 15. The wireless endoscope 15 is hung on the hanger 16 in the state in which the power source switch is off. The standby state is a state in which the battery 22 can be charged, and it is possible to receive setting information.

More specifically, in the standby state, the power receiving portion 24 can charge the battery 22 when the power receiving portion 24 is receiving power supply by radio. Furthermore, in the standby state, the communication I/F 23 enters an operable state in a predetermined cycle under control of the communication controlling portion 21, and the communication controlling portion 21 can execute reception of setting information and a process for storing the setting information in the memory 21a or a process for updating the setting information in the memory 21a.

The operating state is, in FIG. 1, the state of the wireless endoscope 11 the power source switch (not shown) of which has been turned on. The radio communication portion 26, the controlling portion 27, the illuminating portion 28 and the image pickup portion 29 are driven, and an image signal of an endoscopic image can be transmitted to the processor 12 by radio by the radio communication portion 26. Therefore, the processor 12 displays the endoscopic image on the monitor 13, and the wireless endoscope 11 is in a state of being usable in a surgical operation or the like.

That is, the wireless endoscopes 11 and 15 can be in the two states, that is, the operating state and the standby state. When in the operating state, each wireless endoscope can transmit an image signal obtained by the wireless endoscope to the processor 12 from the radio communication portion 26 by radio communication.

Setting information is transmitted from the processor 12 to a wireless endoscope that is in the standby state different from the operating state. In the standby state, each of the wireless endoscopes can receive the setting information from the communication I/F 34 to the communication I/F 23 by radio (or wiredly as described later).

Therefore, in the standby state, though processes for charging the battery 22, receiving setting information and the like are executable, the radio communication portion 26 for radio communication of an image signal of an endoscopic image and the image pickup portion and the illuminating portion for acquiring an endoscopic image are not driven. In the operating state, the radio communication portion 26 for radio communication of an image signal of an endoscopic image, the controlling portion 27, and the illuminating portion 28 and the image pickup portion 29 for acquiring an endoscopic image are driven. Furthermore, in the operating state, setting information about the wireless endoscope 11 is transmitted to the processor 12 via the radio communication portion 26.

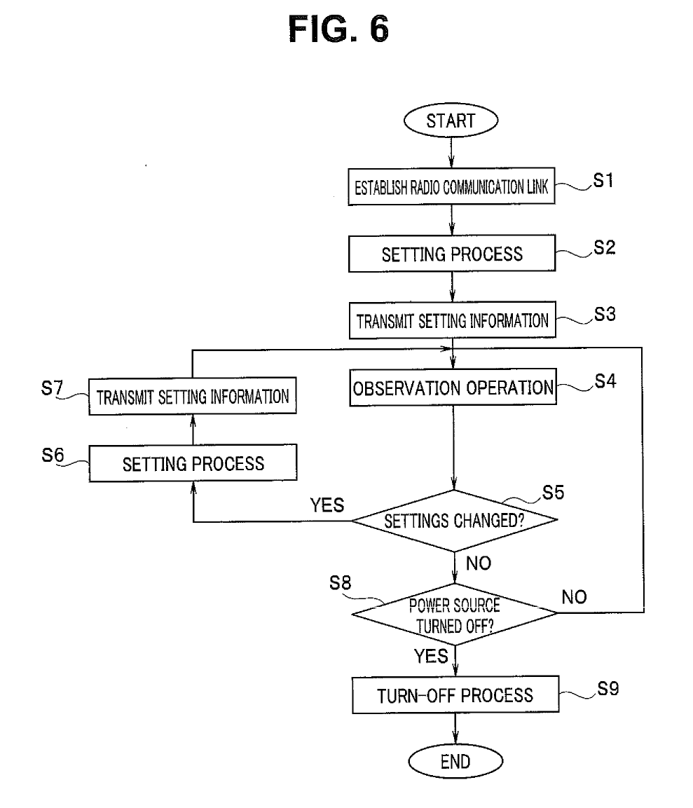

FIG. 6 is a flowchart showing an example of a flow of an operation of the wireless endoscope when the power source of the wireless endoscope is turned on, and the wireless endoscope enters the operating state. Here, description will be made on a case where the power source switch of the wireless endoscope 11 is turned on.

When the wireless endoscope 11 is used for the first time, a process for establishing a radio link with the processor 12 is performed when the power source switch is turned on (step (hereinafter abbreviated as S) 1).

When the wireless endoscope 11 is used, the power source switch of the processor 12 is also turned on, and the processor 12 executes a process for radio connection with the wireless endoscope 11I. Therefore, the wireless endoscope 11 can establish a radio link by the radio connection process of the processor 12. By the radio link being established, a radio channel to be used is decided, and radio channel information is stored in the memory 21a as one piece of setting information. The radio channel information is information about a channel selected from among a plurality of channels for radio communication between the wireless endoscope 11 and the processor 12.

Next, various settings are made by the surgeon, and the controlling portion 27 executes a setting process for the various settings (S2).

The various settings include settings for operation conditions of the wireless endoscope 11, such as settings of allocation of functions to the various operation buttons 27a provided on the operation portion 11b, and setting of operation conditions of the endoscope system 1, such as setting of an observation mode such as a normal light observation mode or a special light observation mode, image processing related settings such as highlight setting, color mode setting and image quality setting.

In the setting process of S2, set setting information is written to the memory 21a and stored.

After the setting process, the controlling portion 27 executes a setting information transmission process for transmitting the various pieces of setting information set at S2 to the processor 12 together with the radio channel information via the radio communication portion 26 (S3).

Note that though, in the present embodiment, the various settings of S1 are made in the wireless endoscope 11 using the various operation buttons provided on the operation portion 11b, all or a part of the various settings of S1 may be made on the operation panel of the processor 12, and setting information about the settings may be transmitted from the processor 12 to the wireless endoscope 11 via the radio communication portions 32 and 26.

Therefore, the setting information set at S2 is held in the memory 21a of the communication controlling portion 21 of the wireless endoscope 11 and is also held in the memory 31a of the controlling portion 31 of the processor 12.

After transmission of the setting information, the wireless endoscope 11 enters a state enabling observation and the like, and the controlling portion 27 executes an observation operation process (S4).

At the time of performing an observation operation, the controlling portion 27 drives the illuminating portion 28 and the image pickup portion 29, generates an image signal of an endoscopic image from a video signal obtained by the image pickup portion 29 and transmits the image signal to the processor 12 via the radio communication portion 26.

During the observation operation, the controlling portion 27 judges whether the surgeon has changed settings or not (S5).

During a surgical operation, the surgeon may change settings of the wireless endoscope 11. When settings are changed (S5: YES), the controlling portion 27 executes a process for setting changed setting information (S6). In the setting process, the setting information is updated, and the changed setting information is held in the memory 21a. The controlling portion 27 executes a setting information transmission process for transmitting the changed setting information to the processor 12 via the radio communication portion 26 (S7).

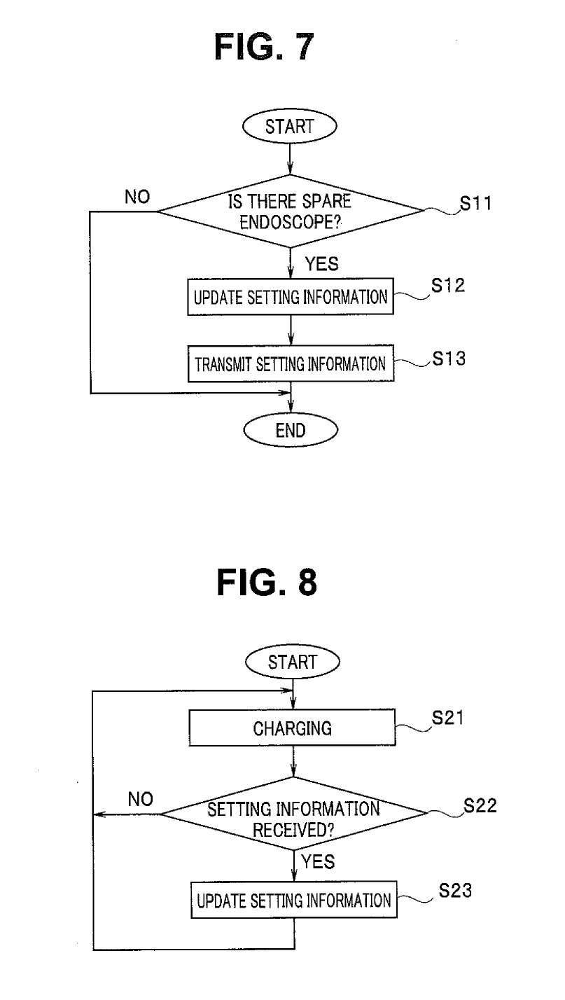

FIG. 7 is a flowchart showing an example of a flow of the setting information transmission process of the processor 12 when setting information about the wireless endoscope that is in the operating state is changed.

When receiving the setting information from the wireless endoscope 11 that is in the operating state by the radio communication portion 32, the controlling portion 31, which is a control device, executes the process of FIG. 7.

The controlling portion 31 judges whether there is a spare wireless endoscope or not (S11). That is, it is judged whether the spare wireless endoscope 15 is hung on the hanger 16 or not.

If there is not a spare wireless endoscope (S11: NO), the controlling portion 31 does no process.

If there is a spare wireless endoscope (S11: YES), the controlling portion 31 executes a setting information update process for writing the changed setting information to the memory 31a (S12) and executes a process for transmitting the changed setting information (S13). The process for transmitting the changed setting information is performed via the communication I/F 34.

As described above, the controlling portion 31 is provided in the processor 12, and the controlling portion 31 stores setting information in the memory 31a and transmits the setting information to the wireless endoscope 15 to store the setting information in the memory 21a of the wireless endoscope 15. The setting information includes at least one of information about a communication channel for radio communication between the wireless endoscope 11 and the processor 12 and information about allocation of functions to the operation buttons of the wireless endoscope 11.

Then, when the wireless endoscope 15 is held by the hanger 16, the controlling portion 31 transmits the setting information to the wireless endoscope 15.

The wireless endoscope 15 that is in the standby state receives the setting information via the communication I/F 23. Furthermore, the communication controlling portion 21 writes the received setting information to the memory 21a or updates the memory 21a.

Therefore, when settings of the wireless endoscope 11 are changed during a surgical operation, changed setting information is held in the memory 21a of the spare wireless endoscope 15 that is in the standby state.

Note that all the setting information set in the wireless endoscope 11 is transmitted to the wireless endoscope 15 at S12, but the setting information includes information to be held only in the processor 12, and, therefore, setting information other than the setting information to be held only in the processor 12 may be transmitted to the wireless endoscope 15.

Returning to FIG. 6, the process transitions to S4 after the process of S7.

If settings have not been changed (S5: NO), the controlling portion 27 judges whether the power source switch of the wireless endoscope 11 has been turned off or not (S8).

When the power source switch of the wireless endoscope 11 has not been turned off (S8: NO), the process transitions to S4.

When the power source switch of the wireless endoscope 11 is turned off (S8: YES), the controlling portion 27 executes a turn-off process for changing the operating state of the wireless endoscope 11 to the standby state (S9).

FIG. 8 is a flowchart showing an example of a flow of an operation of the wireless endoscope that is in the standby state.

When the wireless endoscope 15 is hung on the hanger 16, the power receiving portion 24 of the wireless endoscope 15 that is in the standby state receives power supply by radio, and, therefore, the power receiving portion 24 performs charging (S21).

When in the standby state, the communication controlling portion 21 performs communication with the processor 12 by the communication I/F 23 and judges whether setting information has been received or not (S22).

As described above, in the standby state, the communication controlling portion 21 activates the communication I/F 23 in a predetermined cycle to monitor as to whether communication with the communication I/F 34 of the processor 12 is possible or not. The communication controlling portion 21 activates the communication I/F 23 in the predetermined cycle, and inquires whether or not there is information to be received when communication with the communication I/F 34 of the processor 12 is possible. If there is information to be received, the communication controlling portion 21 receives the information, that is, setting information via the communication I/F 23. As described above, when communication with the communication I/F 34 of the processor 12 is possible, the communication controlling portion 21 can judge whether setting information has been received or not.

If setting information has not been received (S22: NO), the process returns to S21. If setting information has been received (S22: YES), the communication controlling portion 21 executes the setting information update process for storing the received setting information in the memory 21a (S23), and the process returns to S21.

Thus, when settings are changed in the wireless endoscope 11 that is in the operating state, setting information about the changed settings is supplied from the processor 12 to the wireless endoscope 15, and, at each time of change, the changed setting information is stored in the memory 21a of the wireless endoscope 15.

When the battery of the wireless endoscope 11 runs out, the surgeon turns off the power source switch. When the power source switch is turned off, the radio link between the wireless endoscope 11 and the processor 12 is released.

At the time of using the spare wireless endoscope 15, the surgeon turns on the power source switch. When the power source switch of the wireless endoscope 15 is turned on, the operation state of the wireless endoscope 15 is changed from the standby state to the operating state.

The setting information, radio channel information, operation buttons allocation information, image quality setting information and the like that have been already set in the wireless endoscope 11 are read out from the memory 21a in the wireless endoscope 15, and the wireless endoscope 15 can operate in the same setting state as that of the wireless endoscope 11. Therefore, a radio link is established on the same radio channel as that of the wireless endoscope 11, and the surgeon can immediately use the wireless endoscope 15 without re-setting setting information, with the same settings for allocation of functions to the operation buttons and the like as those of the wireless endoscope 11.

As described above, according to the embodiment described above, it is possible to provide a wireless endoscope apparatus enabling, when a wireless endoscope is exchanged for another wireless endoscope, the exchanged other wireless endoscope to be usable in a short time period.

Note that though charging of the wireless endoscope 15 is performed by power supply from the hanger 16 by radio in the embodiment described above, each of the wireless endoscope 15 and the hanger 16 may be provided with a contact for power supply so that charging is performed by wired power supply via the contact.

Moreover, note that though transmission of setting information to the wireless endoscope 15 is performed from the hanger 16 by radio in the embodiment described above, each of the wireless endoscope 15 and the hanger 16 may be provided with a contact for signal transmission so that wired transmission is performed via the contact.

Further, though one wireless endoscope 15 is held by the hanger 16 provided on the cart 14 and is chargeable, and the processor 12 is capable of writing setting information to the wireless endoscope 15 in the embodiment described above, it is also possible that a plurality of spare wireless endoscopes can be held by the hanger 16.

In that case, each of the plurality of wireless endoscopes is charged when the wireless endoscope is held by the hanger 16 and becomes capable of receiving changed setting information and storing the setting information in a memory. Therefore, any of the plurality of wireless endoscopes can be used instead of a wireless endoscope being used.

Furthermore, a display portion may be provided which is configured to, when setting information is written to the memory 21a while the spare wireless endoscope 15 is held by the hanger 16, show that the setting information has been written. For example, the display portion may be caused to blink or light up when setting information is received from the processor 12.

For example, as indicated by two-dot chain lines in FIG. 4, a display portion 41 such as an LED is provided on the operation portion 11b, and the display portion 41 is caused to light up after receiving setting information from the processor 12 and writing the setting information to the memory 21a. The surgeon can recognize that the setting information about the wireless endoscope 11 has been written, by the display portion 41 being lit up.

Furthermore, though the description has been made in the embodiment described above on the assumption that the illuminating portions 28 and the image pickup portions 29 of the wireless endoscopes 11 and 15 are provided on the distal ends of the insertion portions 11a and 15a, respectively, the image pickup portions may be provided on the operation portions 11b and 15b like camera heads. Light sources may be provided on the operation portions so that illuminating light is guided to the distal ends of the insertion portion 11a and 15a by light guides or the like.

Furthermore, though each of the controlling portions 27 of the wireless endoscopes 11 and 15 and the controlling portion 31 of the processor 12 that have been described above includes a CPU and performs the processes of FIGS. 6 and 7 by software, all or a part of the processes may be realized by an analog circuit, a digital circuit such as an FPGA, or the like.

The present invention is not limited to the embodiment described above, and various changes, alterations and the like are possible within a scope that does not depart from the spirit of the present invention.

What is claimed is:

1. A wireless endoscope apparatus comprising:
a first wireless endoscope that can be driven by a first battery;
a second wireless endoscope that can be driven by a second battery;
a receiver capable of radio communication with the first wireless endoscope and the second wireless endoscope;
a first memory provided in the first wireless endoscope and configured to store setting information specifying operation condition of the first wireless endoscope;
a second memory provided in the second wireless endoscope and being capable of storing the setting information;
a third memory provided in the receiver and being capable of storing the setting information; and
a control device provided in the receiver and configured to receive the setting information stored in the first memory from the first wireless endoscope that is in an operating state in which it is possible to pick up an image, via the radio communication and store the setting information in the third memory, and, while the first wireless endoscope is in the operating state, transmit the setting information to the second wireless endoscope that is in a standby state different from the operating state so that the setting information is stored in the second memory.

2. The wireless endoscope apparatus according to claim 1, wherein
the reception of the setting information from the first wireless endoscope and the transmission of the setting information to the second wireless endoscope by the control device are performed each time the operation condition of the first wireless endoscope is set.

3. The wireless endoscope apparatus according to claim 2, wherein
the setting of the operation condition of the first wireless endoscope can be performed by an operation device provided in the first wireless endoscope or the receiver.

4. The wireless endoscope apparatus according to claim 1, wherein
the transmission of the setting information to the second wireless endoscope by the control device is performed for each predetermined cycle.

5. The wireless endoscope apparatus according to claim 1, wherein
the operating state is a state in which a power source of the first wireless endoscope is on, and the standby state is a state in which a power source of the second wireless endoscope is off.

6. The wireless endoscope apparatus according to claim 1, comprising
a holding member configured to hold the second wireless endoscope, wherein
the control device transmits the setting information to the second wireless endoscope when the second wireless endoscope is held by the holding member.

7. The wireless endoscope apparatus according to claim 6, comprising
a charging circuit configured to charge the second battery, wherein
the charging circuit charges the second battery when the second wireless endoscope that is in the standby state is held by the holding member.

8. The wireless endoscope apparatus according to claim 1, wherein
the setting information includes at least one of information about a communication channel for the radio communication between the first wireless endoscope and the receiver and information about allocation of functions to operation buttons of the first wireless endoscope.

9. The wireless endoscope apparatus according to claim 1, wherein
the first and second wireless endoscopes can be in both of the operating state and the standby state.

10. The wireless endoscope apparatus according to claim 1, wherein
the first wireless endoscope comprises a first radio communication circuit for transmitting an image signal of a first endoscopic image acquired by the first wireless endoscope to the receiver by the radio communication;
the receiver comprises a second radio communication circuit for receiving the image signal of the first endoscopic image from the first wireless endoscope by the radio communication;
the receiver comprises a transmitting circuit for transmitting the setting information to the second wireless endoscope; and
the second wireless endoscope comprises a receiving circuit for receiving the setting information from the receiver.

11. The wireless endoscope apparatus according to claim 10, wherein
the first radio communication circuit is capable of transmitting not only the image signal of the first endoscopic image but also the setting information to the receiver by the radio communication.

12. The wireless endoscope apparatus according to claim 10, wherein
the second wireless endoscope comprises a third radio communication circuit for transmitting an image signal of a second endoscopic image acquired by the second wireless endoscope to the second radio communication portion of the receiver by the radio communication;
the second wireless endoscope can be in the operating state and the standby state;
in the operating state, the second wireless endoscope is capable of transmitting the image signal of the second endoscopic image and the setting information from the third radio communication circuit to the receiver, and
in the standby state, the second wireless endoscope is capable of receiving the setting information by the receiving portion wiredly or by radio.

13. The wireless endoscope apparatus according to claim 10, wherein
the transmitting circuit transmits the setting information to the receiving circuit by radio.

14. The wireless endoscope apparatus according to claim 1, wherein
the transmission of the setting information to the second wireless endoscope is performed by near field radio communication.

15. The wireless endoscope apparatus according to claim 1, wherein the receiver is a video processor configured to receive an image signal from the first and second wireless endoscopes, and generate an endoscopic image to be displayed on a monitor.
